(19) United States
(12) Patent Application Publication
(10) Pub. No.: US 2006/0173260 A1
(43) Pub. Date: Aug. 3, 2006

(54) System, device and method for diabetes treatment and monitoring

(75) Inventors: Yuval Gaoni, Zichron Ya'akov (IL); Uri Segev, Ziehron Ya'akov (IL)

Correspondence Address: PEARL COHEN ZEDEK, LLP, 1500 BROADWAY 12TH FLOOR, NEW YORK, NY, 10036, US

(73) Assignee: GMMS LTD

(21) Appl. No.: 11/045,289

(22) Filed: Jan. 31, 2005

(51) Int. Cl. A61B 5/00

(52) U.S. Cl. 600/365; 128/903; 600/300

(57) ABSTRACT

An apparatus and method may provide a system, apparatus and method for monitoring diabetes, including a server to serve data to a cellular communications network; a communications device including a wireless transceiver; a cellular network to enable data to be communicated between the communication device and the server; and a diabetes monitoring device including a wireless transceiver, to enable data to be communicated between the monitoring device and the communication device. The diabetes monitoring device may include sensors for monitoring one or more of for example blood glucose level, physical activity, energy intake and insulin dosage.

### FIELD OF THE INVENTION

The present invention relates to systems, methods and devices useful in monitoring diabetes patients. Specifically, embodiments of the present invention relate to systems, methods and apparatuses that provide for example enhanced blood glucose level monitoring, energy consumption monitoring, and insulin dosage monitoring for diabetes patients.

### BACKGROUND OF THE INVENTION

Diabetes is a growing health problem across the world, and in the United States in particular it has risen about six-fold since 1950, now affecting approximately 16 million Americans. About one-third of those 16 million do not know that they have the disease. Diabetes-related health care costs total nearly $100 billion per year and are increasing. Diabetes contributes to over 200,000 deaths each year.

There are currently no cures for diabetes; however, the disease may be treated and managed successfully by closely monitoring and managing ones blood-glucose levels through exercise, diet and medications. For Type 1 diabetes, where a person lacks insulin, insulin is typically administered several times each day, for example, around meals to cope with the glucose load from digestion. A type 1 diabetes patient should monitor their blood-glucose levels several times a day and adjust the amounts of insulin that are to be injected accordingly. This keeps one's blood-glucose concentration from fluctuating wildly.

To monitor blood glucose, patients may make use of a number of commercial blood-glucose monitors. The process of monitoring typically involves reacting a test strip with a drop of blood (e.g., finger prick). The glucose in the blood reacts chemically with an enzyme on the test strip called glucose oxidase, and the product of the reaction, gluconate, combines with another chemical to make the strip turn blue. The device generally measures the degree of color change to determine and display the concentration of glucose in the blood sample. In other glucose monitoring devices the test strip may serve as a platform for other substances to chemically react with the glucose in the blood, resulting in an electrical resistance and/or electrical current which reflects the blood glucose level.

Blood-glucose monitoring generally requires extensive user involvement, including the administration of measurements and treatments, and keeping of updated logs. The administration of such actions may often lead to a reduction in quality of life, and it is often the case that patients keep only partial and incomplete logs, often leading to complications.

### SUMMARY OF THE INVENTION

There is provided, in accordance with an embodiment of the present invention, an apparatus, system, and method for diabetes monitoring and treatment, including patient parameter monitoring with automated analyzing and/or reporting. For example, the apparatus may enable automated reporting to a medical care center, thereby freeing the patient of this chore and increasing the accuracy of the data.

According to some embodiments of the present invention, patient parameter monitoring may include one or more of blood glucose level monitoring, physical activity monitoring, medication dosage monitoring, and energy intake monitoring.

In one embodiment a system for monitoring diabetes is provided that includes a server to serve data to a communications network, a communications device including a wireless transceiver and communications mechanism, a cellular network to enable data to be communicated between the communication device and the server, and a diabetes monitoring device including a wireless transceiver, to enable data to be communicated between the monitoring device and the communication device.

In one embodiment a patient monitoring apparatus is provided that includes a computing device, a blood glucose level sensor to enable measurement of blood glucose levels of a patient, a power source, a real time clock to provide the time at which measurements were performed, and a wireless transceiver to wirelessly transmit measurement data to an external communications device.

In one embodiment a patient monitoring apparatus is provided that includes a computing device, a physical activity level sensor to enable measurement of energy consumption of a patient, a power source, a real time clock to provide the time at which measurements were performed, and a wireless transceiver to wirelessly transmit measurement data to an external communications device.

In one embodiment a patient monitoring apparatus is provided that includes a computing device, a blood glucose level sensor to enable measurement of blood glucose levels of a patient, a physical activity level sensor to enable measurement of energy consumption of a patient, a power source, a real time clock to provide the time at which measurements were performed, and a wireless transceiver to wirelessly transmit measurement data to an external communications device.

In one embodiment a medication administration monitoring device is provided that may include a computing device, a medication administration element, a medication dosage sensor to enable measurement of medication administered to a patient, a power source, a real time clock to provide the time at which dosages of medication were administered and/or measurements of dosages administered were performed, and a wireless transceiver to wirelessly transmit measurement data to an external communications device.

According to another embodiment of the present invention a method for monitoring diabetes is provided, that includes measuring one or more patient parameters using a diabetes monitoring and treatment device, transmitting data between the diabetes monitoring and treatment device and a communication device using a wireless transceiver, and processing the received measurement data by the communications device.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic diagram of a diabetes monitoring system including a diabetes monitoring device, according to some embodiments of the present invention;

FIG. 2 is a schematic block diagram of the diabetes monitoring device of FIG. 1, according to some embodiments of the present invention;

FIG. 3 is a schematic block diagram of the diabetes monitoring device of FIG. 1, according to some embodiments of the present invention;

FIG. 4 is a schematic block diagram of the integrated diabetes monitoring device of FIG. 1, according to some embodiments of the present invention;

FIG. 5 is a diagram illustrating the components of the integrated diabetes monitoring device of FIG. 4, according to some embodiments of the present invention;

FIG. 6 is a schematic block diagram of the diabetes monitoring device of FIG. 1, according to some embodiments of the present invention;

FIG. 7 is a flowchart illustrating a method for monitoring diabetes according to some embodiments of the present invention; and

FIG. 8 is a flowchart illustrating a method for monitoring diabetes according to some embodiments of the present invention.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

Embodiments of the present invention enable improved diabetes monitoring and/or treatment, including patient parameter monitoring with automated data collection, transmission, analysis, reporting, generation of alerts and other suitable monitoring and/or treatment functions.

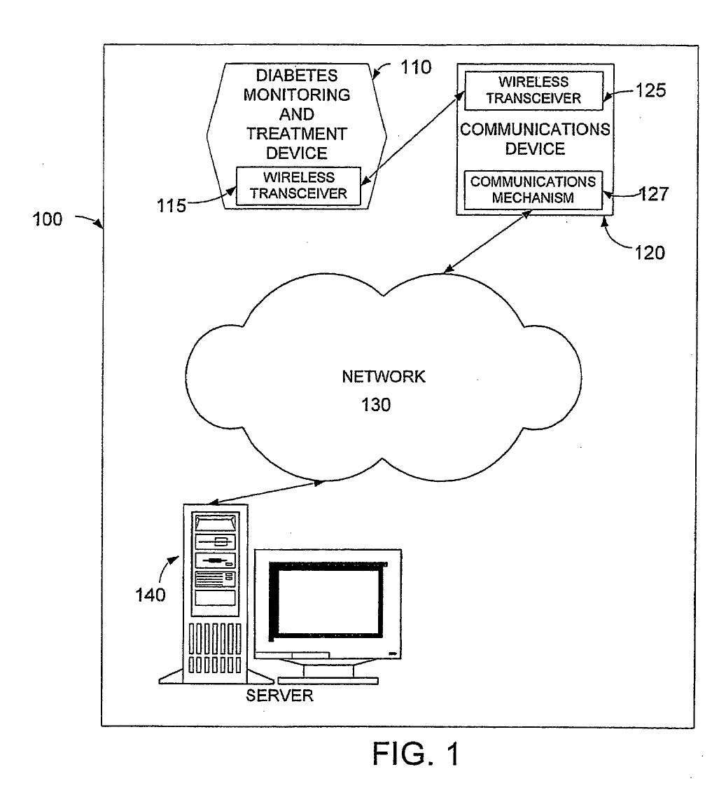

Reference is now made to FIG. 1, which is a schematic block diagram illustration of a diabetes monitoring and treatment system 100 including a diabetes monitoring and treatment device 110, according to some embodiments of the present invention. Device 110 may be a wireless device that may be attachable to a patient or wearable. Device 110 may be transported along with a patient to enable measurement of one or more selected parameters according to the need. Device 110 may operate according to one or more wireless data communications standards, for example, including any suitable wireless personal area network technology or wireless local area network technology. For example, device 110 may include a wireless communication transceiver, circuit or chip 115, for example, a Bluetooth transceiver, Infrared transceiver (e.g., IrDA), Unlicensed Broadband Wireless (UBW) transceiver, WiFi transceiver, ZigBee transceiver or other suitable wireless communications transceiver, circuit, transponder or adapter, to enable device 110 to communicate with external communications devices (e.g., 120). Wireless transceiver 115 may support point-to-point and/or multipoint applications.

System 100 may include a communications device 120, for example a cellular phone, personal communicator, pager, mobile computer, laptop computer, watch, or other suitable communication or computing device. Communications device 120 may include a wireless transceiver 125 to enable device 120 to receive and/or transmit measurement data from/to monitoring and treatment device 110 Communications device 120 may enable wireless data communications using one or more wireless communications technologies, for example, including any suitable wireless personal area network technology or wireless local area network technology. For example, wireless transceiver 125 may include one or more of a Bluetooth transceiver, Infrared transceiver (e.g., IrDA), Unlicensed Broadband Wireless (UBW) transceiver, WiFi transceiver, ZigBee transceiver or other suitable wireless communications transceivers, circuits, transponders or adapters, to enable communications device 120 to communicate with additional devices (e.g., monitoring and treatment device 110).

Communications device 120 may include at least one communications mechanism 127 to enable communication over a public and/or private network. Communications mechanism 127 may enable, for example, communications device 120 to communicate with other devices or systems using wire based and/or wireless communications mechanisms. For example, communications mechanism 127 may enable communications device 120 to communicate with a medical center, medical specialist, monitoring authority, responsible guardian etc. using one or more of cellular, WiFi, cable, ADSL or other suitable data transmission technologies. Communications device 120 may have at least one of a processor, data storage, display mechanism, keyboard, program memory and power source to enable data entry, data processing, analysis, and display etc.

Communications device 120 may include at least one software application capable to process measurement or other data received from device 110. Such application software may perform data manipulation and may determine various operational aspects of device 120. Communications device 120 may include one or more subprograms, units or modules that may help, for example, implement one or more selected algorithms used to create and manage a database, analyze data, generate and implement reports, warnings and/or alerts, recommend specific actions, display data on a Graphical User Interface (GUI), convey information from the database to a server 140 using, for example, a cellular network, interact with monitoring and treatment device 110, and/or perform other suitable functions. According to some embodiments of the present invention, communications device 120 may include at least one software application capable of receiving data from a plurality of measurement sensors or circuits in device 110, and to process the received data according to the sensor at which the particular data was measured. Communications device 120 may include a mechanism and suitable application software for manually entering data, for example, energy intake data or other suitable data. One or more communication standards may be used, for example, Bluetooth, infrared, Wi-Fi etc.

In one example, communications device 120 may send a signal to monitoring and treatment device 110 to perform a measurement, test, calibration etc., and optionally to transmit the results back to communications device 120. Communications device 120 may be set up and operated on a per user basis. For example, a user or medical care center may configure the device to implement alerts, alarms, reminders, advice, messaging preferences, communication preferences, and information sharing preferences etc. for a patient, for example, according to a patient's privacy demands, health profile etc. Communications device 120 may be an off-the-shelf cellular phone, pager device, mobile computing device or other suitable device that may be adapted for usage according to embodiments of the present invention. In some embodiments the communication device 120 input and/or output elements (e.g., screen, keypad, microphone, speaker, etc.) may be used to control device 110 and/or display, interact with and otherwise utilize the data received from device 110 or from server 140.

System 100 may include a network 130, for example, a cellular communications network, Internet, intranet, wireless network and/or one or more other suitable communications networks. System 100 may include a server 140, or another suitable data serving system. Server 140 may include one or more workstations, and may include at least one data processor, database, output device, input device, communications facilities etc. Server 140 may serve data to multiple users via network 130, or may provide other suitable service functions. In one embodiment server 140 may be a central server to receive data from and/or provide data to multiple user devices. In other embodiments server 140 may be a plurality of distributed servers.

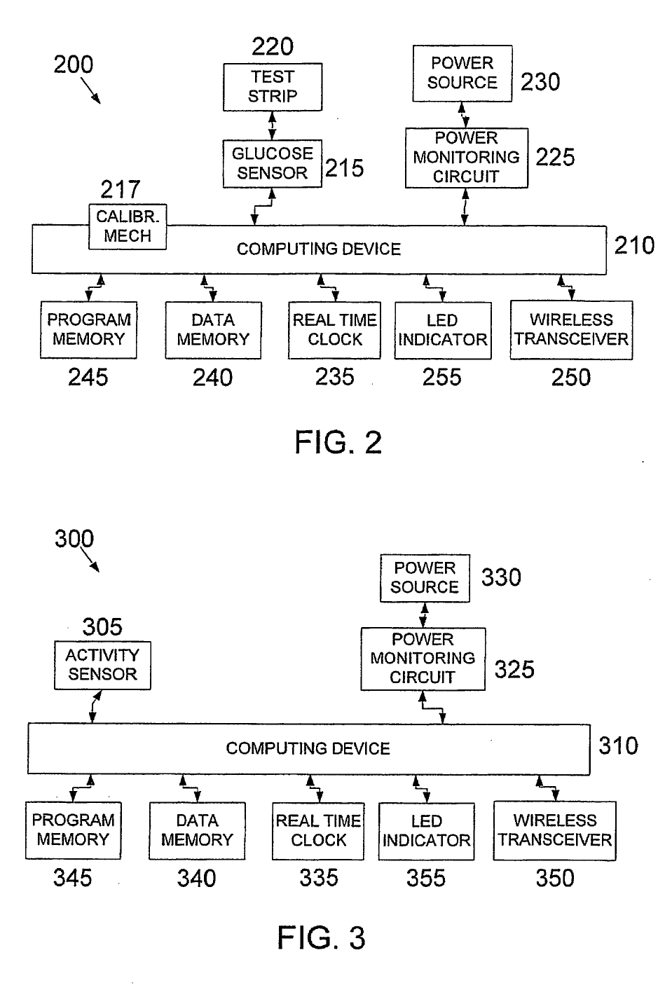

Reference is now made to FIG. 2, which depicts a diabetes monitoring device 200 according to some embodiments of the present invention, which is adapted to measure blood glucose levels and transmit data wirelessly to communications device 120. Device 200 may include a computing device 210, for example, a state machine, controller, CPU or other suitable device to control operations of device 200. Device 200 may include a measurement circuit or sensor 215 for measurement of blood glucose levels from a test strip 220 or other suitable blood glucose level measurement apparatus. For example, computing device 210 may constantly or periodically read the electrical current levels generated by test strip 220 when a drop of blood is applied to test strip 220 and strip 220 is inserted into sensor 215. These current levels may be translated into blood glucose values by, for example, algorithms implemented in the software (e.g., firmware) being run by computing device 210. In other embodiments the translation of current levels into blood glucose values may be executed by communications device 120. Device 200 may include, for example, a glucose test strip insertion orifice. Device 200 may include a calibration mechanism 217, for example, an orifice or other suitable mechanism for calibrating device 200. Device 200 may include a power source 225 (e.g., battery), and may include a power monitoring circuit 230 to monitor power level of device 200.

Device 200 may include a Real Time Clock (RTC) 235 to provide a timer for determining the timing of various operations performed or due to be performed. Device 200 may include a data memory to include data from one or more measurements, and other suitable data. In one example, data memory 240 may maintain data for a plurality of measurements, to facilitate maintenance of measurement data even if a transmission channel for transferring data to communications device 120 is unavailable for an extended or selected period of time. For example, time of measurement and/or the measured blood glucose values may be temporarily stored in data memory 240. These values may be scheduled for transmission to communications device 120, where the data may be stored in a local database and further processed, displayed, analyzed, transmitted etc. Device 200 may include a program memory 245 to store program code for device 200. Device 200 may include a wireless communications transceiver 250, for example, a Bluetooth transceiver, Infrared transceiver (e.g., IrDA), Unlicensed Broadband Wireless (UBW) transceiver, WiFi transceiver, ZigBee transceiver or other suitable wireless communications transceiver, circuit, transponder or adapter. Device 200 may include a LED indicator 255, or other suitable indicator to help indicate the status or functioning of device 200. For example, LED indicator 255 may flash as long as connection with the communication device 120 is not available For example, transceiver 250 may attempt to connect to the communication device 120 repeatedly, and when such a connection is established, the local data may be transferred, the data in memory 240 may be deleted, and indicator LED 255 may be turned off.

Device 200 may facilitate manual and/or automatic transfer of measurement data to a communications device 120, such that the relevant processing, displaying, data entry, analyzing and transmission functions etc. for device 200 may be delegated to communications device 120. In this way device 200 may function as a simplified blood glucose level measurement device which may operate together with communications device 120 to provide at least the full functionality of a standard blood glucose level monitoring device. For example, device 200 may not require one or more of power monitoring circuit 230, program memory 245, and LED indicator 255, which may be replaced partially or completely in communications device 120. Furthermore, device 200 may not require one or more of, for example, a screen, keypad, database, CPU etc., which may instead be provided by communications device 120. Communications device 120 may utilize, for example, a screen, keypad, database, CPU etc., to operate device 200 and/or interact with device 200, by executing code from one or more applications, for example a diabetes monitoring application, configured on communications device 120.

Reference is now made to FIG. 3, which depicts a diabetes monitoring device 300 according to some embodiments of the present invention, which is adapted to measure for example physical activity levels or body energy consumption of a patient or user and transmit data wirelessly to communications device 120. Device 300 may include a computing device 310, for example, a state machine, controller, CPU or other suitable device to control operations of device 300. Device 300 may include a physical activity measurement circuit or sensor 305, for example, a step sensor (e.g., accelerometer) or other suitable measurement circuits, to measure a user's physical activity. For example, an electronic pendulum based sensor, mechanical pendulum based sensor, gas based acceleration measurement sensor, a solid-state integrated circuit (e.g., MEMS based) device or other suitable sensor devices may be used. In one example, sensor 305 may operate based on the temperature gradient principle. For example, computing device 310 may constantly or periodically read the acceleration values generated by sensor 305. The consumed energy values may be calculated by, for example, algorithms implemented in the software (e.g., firmware) being run by computing device 310. In other embodiments the calculation of consumed energy values may be executed by communications device 120. Device 300 may be calibrated, for example, by linking the number of steps taken by an individual to the distance covered. Other suitable mechanisms for calibration device 300 may be used. Device 300 may include a power source 325 (e.g., battery), and may include a power monitoring circuit 330 to monitor power level of device 300.

Device 300 may include a Real Time Clock (RTC) 335 to provide a timer for determining the timing of various operations performed or due to be performed. Device 300 may include a data memory to include data from one or more measurements, and other suitable data. In one example, data memory 340 may maintain data for a plurality of measurements, to facilitate maintenance of measurement data even if a transmission channel for transferring data to communications device 120 is unavailable for an extended or selected period of time. For example, time of measurement and/or the measured acceleration values may be temporarily stored in data memory 340. These values may be scheduled for transmission to communications device 120, where the data may be stored in a local database and further processed, displayed, analyzed, transmitted etc. Device 300 may include a program memory 345 to store program code for device 300. Device 300 may include a wireless communications transceiver 350, for example, a Bluetooth transceiver, Infrared transceiver (e.g., IrDA), Unlicensed Broadband Wireless (UBW) transceiver, WiFi transceiver, ZigBee transceiver or other suitable wireless communications transceiver, circuit, transponder or adapter. Device 300 may include a LED indicator 355, or other suitable indicator to help indicate the status or functioning of device 300. For example, LED indicator 355 may flash as long as connection with the communication device 120 is not available. For example, transceiver 350 may attempt to connect to the communication device 120 repeatedly, and when such a connection is established, the local data may be transferred, the data in memory 340 may be deleted, and indicator LED 355 may be turned off.

Device 300 may facilitate manual and/or automatic transfer of measurement data to a communications device 120, such that the relevant processing, displaying, analyzing and transmission functions etc. for device 300 may be delegated to communications device 120. In this way device 300 may function as a simplified physical activity level measurement device which may operate together with communications device 120 to provide at least the full functionality of a standard physical activity level measurement device. For example, device 300 may not require one or more of power monitoring circuit 330, program memory 345, and LED indicator 355, which may be replaced partially or completely in communications device 120. Furthermore, device 300 may not require one or more of, for example, a screen, keypad, database, CPU etc., which may instead be provided by communications device 120. Communications device 120 may utilize, for example, a screen, keypad, database, CPU etc., to operate device 300 and/or interact with device 300, by executing code from one or more applications, for example a diabetes monitoring application, configured on communications device 120.

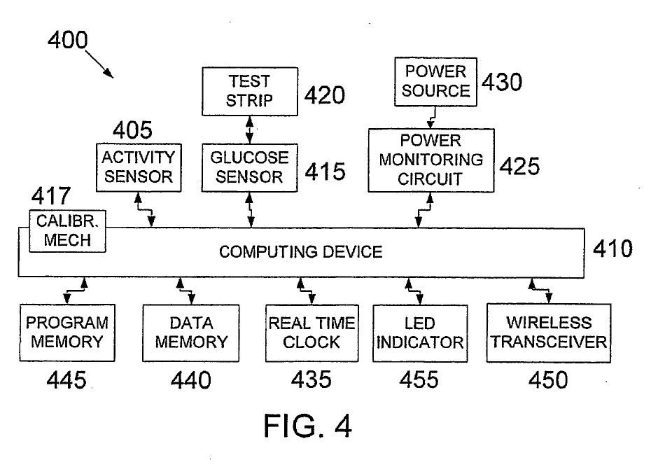

Reference is now made to FIG. 4, which depicts an integrated diabetes monitoring device 400 according to some embodiments of the present invention, which is capable of measuring for example blood glucose level and physical activity or body energy consumption of a patient or user and transmitting data wirelessly to communications device 120. Device 400 may include a computing device 410, for example, a state machine, controller, CPU or other suitable device to control operations of device 400. Device 400 may include a measurement circuit or sensor 415 for measurement of blood glucose levels from a test strip 420 or other suitable blood glucose level measurement apparatus. Device 400 may include a physical activity measurement circuit or sensor 405, for example, a step sensor or other suitable measurement circuits, to measure a user's physical activity. For example, an electronic and/or mechanical pendulum based sensor, gas based acceleration measurement sensor, a solid-state integrated circuit (e.g., MEMS based) device or other suitable sensor devices may be used. In one example, sensor 305 may operate based on the temperature gradient principle. For example, computing device 410 may constantly or periodically read the electrical current levels generated by test strip 420 when a drop of blood is applied to test strip 420 and strip 420 is inserted into sensor 415, and/or the acceleration values generated by sensor 405. The current levels may be translated into blood glucose values by, for example, algorithms implemented in the software (e.g., firm-ware) being run by computing device 410. Additionally, the consumed energy values may be calculated by, for example, algorithms implemented in the software (e.g., firmware) being run by state machine 410. In other embodiments the translation of current levels into blood glucose values and/or the calculation of consumed energy values may be executed by communications device 120. Device 400 may include a calibration mechanism 417, for example, an orifice or other suitable mechanism for calibrating device 400. Device 400 may include a power source 425 (e.g., battery), and may include a power monitoring circuit 430 to monitor power level of device 400.

Device 400 may include a Real Time Clock (RTC) 435 to provide a timer for determining the timing of various operations performed or due to be performed. Device 400 may include a data memory to include data from one or more measurements, and other suitable data. In one example, data memory 440 may maintain data for a plurality of measurements, to facilitate maintenance of measurement data even if a transmission channel for transferring data to communications device 120 is unavailable for an extended or selected period of time. For example, time of measurement and/or the measured blood glucose values and/or acceleration values may be temporarily stored in data memory 440. These values may be scheduled for transmission to communications device 120, where the data may be stored in a local database and further processed, displayed, analyzed, transmitted etc. Device 400 may include a program memory 445 to store program code for device 400. Device 400 may include a wireless communications chip 450, for example, a Bluetooth transceiver, Infrared transceiver (e.g., IrDA), Unlicensed Broadband Wireless (UBW) transceiver, WiFi transceiver, ZigBee transceiver or other suitable wireless communications transceiver, circuit, transponder or adapter. Device 400 may include a LED indicator 455, or other suitable indicator to help indicate the status or functioning of device 400. For example, LED indicator 455 may flash as long as connection with the communication device 120 is not available. For example, transceiver 450 may attempt to connect to the communication device 120 repeatedly, and when such a connection is established, the local data may be transferred, the data in memory 440 may be deleted, and indicator LED 455 may be turned off.

Device 400 may facilitate automatic transfer of measurement data to a communications device 120, such that the relevant processing, displaying, analyzing and transmission functions etc. for device 400 may be delegated to communications device 120. In one embodiment an “on” button may initiate data transfer to communications device 120, for example, to help save power. In this way device 400 may function as a simplified physical activity level measurement device which may operate together with communications device 120 to provide at least the full functionality of a standard physical activity level measurement device. For example, device 400 may not require one or more of power monitoring circuit 430, program memory 445, and LED indicator 455, which may be replaced partially or completely in communications device 120. Furthermore, device 400 may not require one or more of, for example, a screen, keypad, database, CPU etc., which may instead be provided by communications device 120.

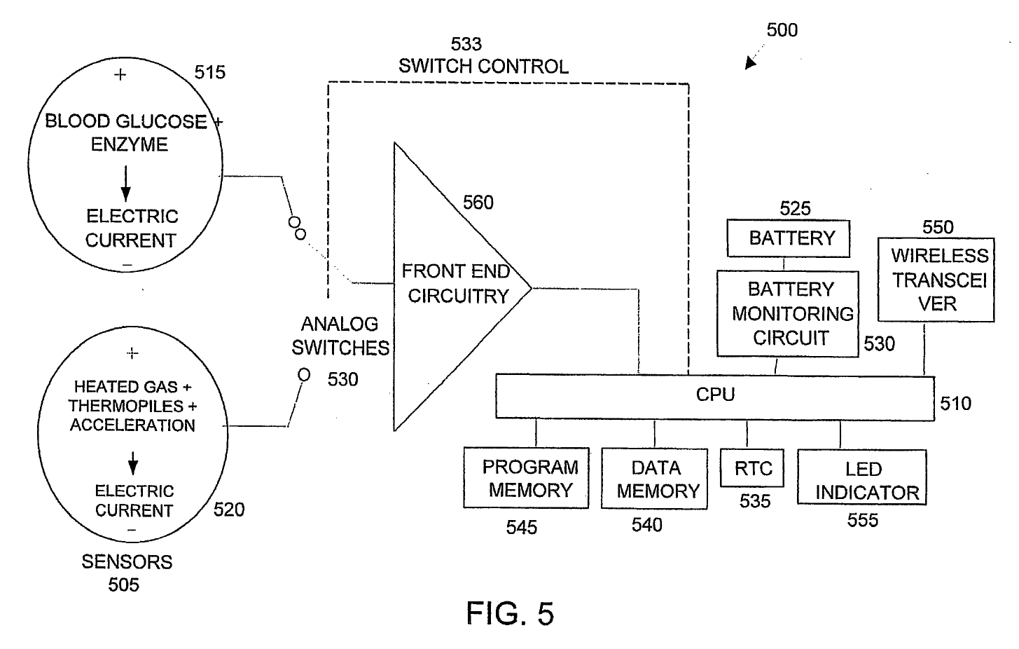

Reference is now made to FIG. 5, which is a diagram illustrating the components of an integrated diabetes monitoring device 500, which may include device 400 of FIG. 4, according to some embodiments of the present invention. Device 500 may include an integrated sensors sub-system 505 that may include, for example, blood glucose level sensor 515 and physical activity or consumed energy sensor 520 (erg., acceleration sensor), connected individually to a joint or unified front end circuitry 560. Front-end circuitry 560 may process the analog signals received from sensors sub-system 505 and may convert these signals to digital signals for, for example, further processing and/or transmission. The selection of a sensor (e.g., 515 or 520) for processing may be made by CPU 510 and its associated software, for example, through Switch Control signal 533. CPU 510 may generate switch control signals 533. Integrated sensors sub-system 505 may be implemented, for example, in an integrated circuit (IC). Device 500 may include a plurality of analog switches 530 to facilitate receiving of analog signals from sensors 515 and 520, to transmit the signals to front-end circuitry 560. Since in both sensors 515 and 520 electrical resistance and/or electrical currents may be altered in response to specific blood glucose levels and/or acceleration values respectively, the measurement and signal conditioning electrical circuitry in the front-end circuitry 560 may be the same for both sensors. Additionally, an array of analogue switches 530 may be used to connect front-end circuitry 560 to sensors 515 and 520, to enable current to flow from either sensor 515 or sensor 520 to front-end circuitry 560, respectively, at a selected moment. In this way, for example, the functions of both activity sensing and blood glucose level sensing may be integrated into at least one state machine or controller. Joint front-end circuitry 560 may enable joint usage of, for example operational amplifiers, comparators and filters for handling analog signals from all sensor elements (e.g., 515, 520).

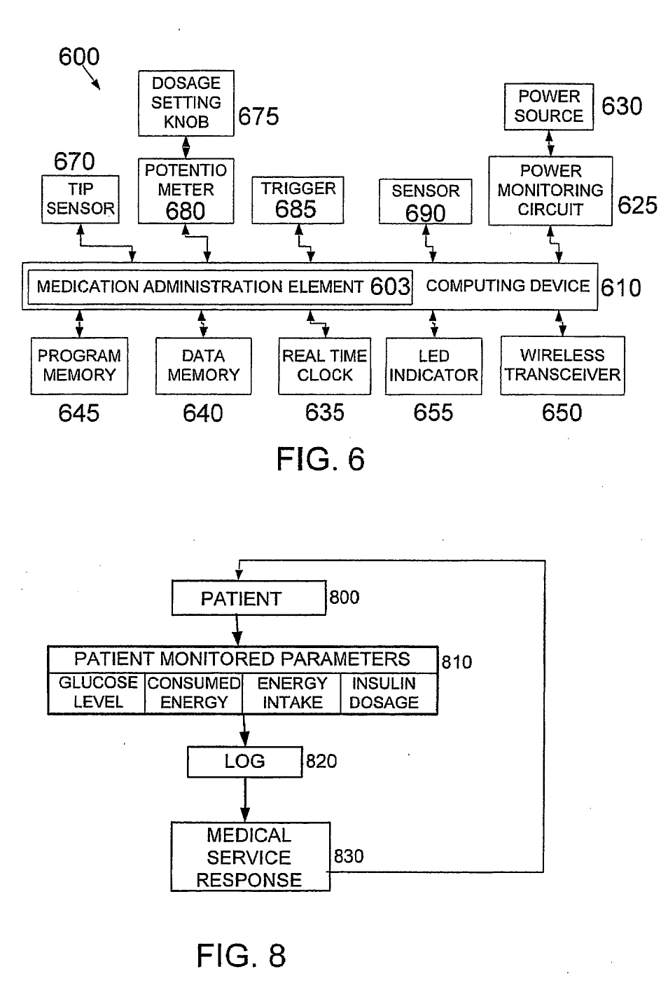

Reference is now made to FIG. 6, which depicts a medication monitoring and administration device 600 according to some embodiments of the present invention, which is adapted to enable administration of medication, for example insulin, to a patient, automated monitoring of treatment for the patient, and automated and/or manual transmission of data wirelessly to communications device 120. Device 600 may include a medication administration element 603, for example, an insulin syringe, pen-like device, pump etc. Device 600 may include a computing device 610, for example, a state machine, controller, CPU or other suitable device to control operations of device 600. Device 600 may include a medication dosage sensing element, to determine, for example, the type of medication administered using device 600, the dosage set by the user, and the time and date of one or more actual injections or treatments. For example, an insulin dosage-sensing element may be used to determine, for example, the type of insulin administered etc. Device 600 may include, for example, a tip sensor 670, to sense pressure or skin contact on the tip of the pen (e.g., a conductive plate on the tip may be used in tandem with a similar plate on a trigger to sense the capacitance change at the moment of the injection, or a pressure sensor on the tip). Device 600 may include, for example, a potentiometer or encoder 680 that may be co-assembled with a dosage setting knob 675 to read the dosage setting. Device 600 may include, for example, a trigger sensor 685 (e.g., a momentary switch implemented by a pressure sensor, opto-mechanical or opto-electronic device etc.) that may signal the moment of injection. Device 600 may include at least one sensor system 690 to sense the type of the medication administered. For example, sensor 690 may include one or more of a color sensor, tactile sensor, a conductive element sensor and/or other suitable sensors to identify a medication. In one example sensor 690 may enable identification of an insulin vile and/or cartridge, to identify the insulin type loaded into device 600. Device 600 may include a power source 625 (e.g., battery), and may include a power monitoring circuit 630 to monitor power level of device 600.

Device 600 may include a Real Time Clock (RTC) 635 to provide a timer for determining the timing of various operations performed or due to be performed, for example, to provide the time at which dosages of insulin were administered and/or measurements of dosages administered were performed. Device 600 may include a data memory to include data from one or more operations performed, and other suitable data. In one example, data memory 640 may maintain data for a plurality of operations performed, to facilitate maintenance of data even if a transmission channel for transferring data to communications device 120 is unavailable for an extended or selected period of time. Device 600 may include a program memory 645 to store program code for device 600. Device 600 may include a wireless communications transceiver 650, for example, a Bluetooth transceiver, Infrared transceiver (e.g., IrDA), Unlicensed Broadband Wireless (UBW) transceiver, WiFi transceiver, ZigBee transceiver or other suitable wireless communications transceiver, circuit, chip, transponder or adapter. Device 600 may include a LED indicator 655, or other suitable indicator to help indicate the status or functioning of device 600.

Device 600 may facilitate automatic transfer of measurement data to a communications device 120, such that the relevant processing, displaying, analyzing and transmission functions etc. for device 600 may be delegated to communications device 120 may perform. In this way device 600 may function as a simplified insulin administration device as it may operate together with communications device 120 to provide at least the fill functionality of a standard insulin administration device- For example, device 600 may not require one or more of power monitoring circuit 630, program memory 645, and LED indicator 655, which may be replaced partially or completely in communications device 120.

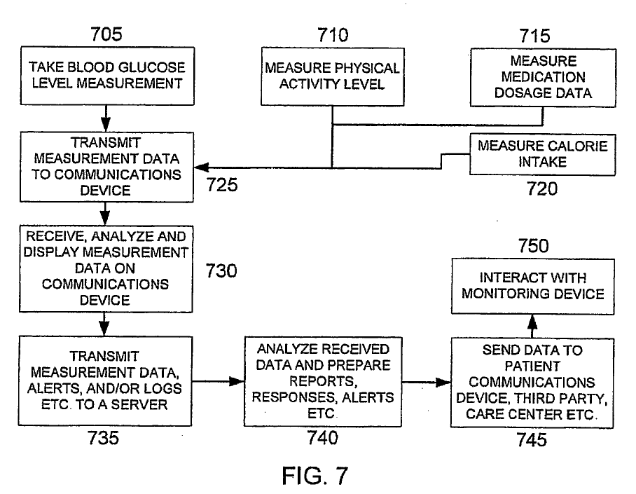

Reference is now made to FIG. 7 which schematically illustrates a series of operations or processes that may be implemented to enable monitoring and/or treatment of one or more diabetes parameters, according to some embodiments of the present invention. At block 705 a patient's blood glucose level may be measured, for example by monitoring and treatment device 110, or by another suitable device. At block 710 a patient's consumed energy (e.g., physical activity performed) may be measured by a device such as monitoring and treatment device 110. At block 715 a patient's insulin dosage data may be measured by device 110. At block 720 a patient's energy intake level (e.g., calorie intake) may be measured by monitoring and treatment device 110. Other parameters or combinations of parameters may be measured.

At block 725 measurement data from one or more parameters may be transmitted, for example to communications device 120 or another suitable device. For example, data from monitoring and treatment device 110 may be continuously received by the communications device 120, using a wireless communications transceiver, for example, a Bluetooth transceiver, Infrared transceiver (e.g., IrDA), Unlicensed Broadband Wireless (UBW) transceiver, WiFi transceiver, ZigBee transceiver or other suitable wireless communications transceiver.

At block 730 communications device 120 may receive, analyze, display etc. measurement data. Communications device 120 may, for example, add the data received to a database or data memory. For example, such a database may be constantly or periodically analyzed according to a user's profile. For example, patterns of a user's blood glucose levels, physical activity, insulin dosage patterns and energy intake may be created, maintained and utilized. According to one example, upon reception of new data from monitoring and treatment device 110, and/or a user manual entry, communications device 120 may compare inputted data against a user profile and/or against absolute parameter thresholds. For example, communications device 120 may detect exceptional values for blood glucose levels, physical activity levels, insulin dosage levels and/or energy intake levels on a per user basis.

At block 735 communications device 120 may transmit measurement data, alerts, logs or other suitable data to a server 140, for example, at a medical center. At block 740 server 140 may analyze or otherwise handle data received from communications device 120. Server 140 may generate reports, responses, alerts etc. At block 745 server 140 may send data or commands etc. to one or more communications devices 120, for example, a patient's communications device 120 and/or third party communications device (e g., a parent, care giver, care center, physician, pharmacy etc.). For example, server 140 may send a message with data, an alert and/or one or more commands via SMS to one or more selected destinations. In some embodiments server 140 may send data directly to one or more monitoring and treatment devices 110. Upon receipt of a message or data etc. communication device 120 may display tile data, implement the alert, and/or execute the command(s) etc., for example, as at block 730. Other types of messages with additional or alternative contents may be sent by server 140.

At block 750 communications device(s) 120 may interact with the patient, third party and/or monitoring and treatment device 110. For example, communications device(s) 120 may send an alert to a patient or third party to suggest a line of action to take. For example, communications device(s) 120 may send a signal to monitoring and treatment device 110 to perform a measurement, test, calibration etc., and optionally to transmit the results back to communications device(s) 120. Other interactive functions may be implemented. Any combination of the above steps may be implemented. Further, other steps or series of steps may be used,

Reference is now made to FIG. 8, which is a flowchart illustrating a method for monitoring diabetes. The method may be performed using system 100 of FIG. 1, according to some embodiments of the present invention, but other suitable devices, such as those described herein, or other devices, may be used. In operation 800 one or more devices 110 may be used by a patient to provide measurements of a plurality of patient parameters. In operation 810 device 110 may perform measurements of one or more patient parameters, thereby enabling, for example, improved monitoring of multiple parameters for diabetes patients. For example, device 400 may enable monitoring and reporting of one or more of a patient's glucose level, a patient's physical activity level, and other suitable patient parameters. For example, device 110 may enable monitoring and reporting of one or more of a patient's glucose level, physical activity level, insulin usage, and energy intake level. It will be appreciated that additional parameter sensors nay be applied on or integrated with device 110, for achieving additional functions. Furthermore, additional modes of operation may be implemented, and additional device components and/or dimensions may be applied. Other steps or series of steps may be used.

In operation 820 data from one or more measurements may be automatically logged by device 110, and may be stored in device 110, 120 and/or 140, and/or transmitted immediately to a care center, medical service etc. A medical service may receive, process and analyze the data, and prepare a response or course of action in response to the data For example, the medical service may prepare advice, warnings, alerts etc. for the patient in accordance with the measurement data received. The medical service may send such a response to the patient, for example, via the patient's communication device, and/or to other designated destinations.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A system for monitoring diabetes, the system comprising:
a server to serve data to a communications network;
a communications device, said communications device including at least a wireless transceiver and a communications mechanism;
a network to enable data to be communicated between said communication device and said server; and
a diabetes monitoring device, said monitoring device including at least a wireless transceiver, to enable data to be communicated between said monitoring device and said communication device.

2. The system of claim 1, wherein said transceiver operates according to one or more wireless communications standards selected from the group consisting of a Bluetooth transceiver, Infrared transceiver, Unlicensed Broadband Wireless (UBW) transceiver, WiFi transceiver and ZigBee transceiver.

3. The system of claim 1, wherein said communications device is selected from the group consisting of cellular phone, personal communicator, pager, mobile computer, and watch.

4. The system of claim 1, wherein said communications device includes a mechanism and application software for manually entering data.

5. The system of claim 1, wherein said communications device is to display data received from said diabetes monitoring device,

6. The system of claim 1, wherein said communications device is to analyze data received from said diabetes monitoring device.

7. The system of claim 1, wherein said communications device is to display analysis results.

8. The system of claim 1, wherein said communications device is to facilitate data entry for the system.

9. The system of claim 1, wherein said communications device is to provide an alert as a response to data received from said diabetes monitoring device.

10. The system of claim 1, wherein said network is selected from one or more of the group consisting of a wire based network and a wireless network.

11. An apparatus for monitoring diabetes, the apparatus comprising:
a computing device;
a blood glucose level sensor to enable measurement of blood glucose levels of a patient;
a power source;
a real time clock to provide the time at which measurements were performed; and
a wireless transceiver to wirelessly transmit measurement data to an external communications device.

12. The apparatus of claim 11, wherein said communications device is to receive data from said monitoring device, and is to implement one or more of processing data, displaying data, analyzing data, entering data and transmitting data to said server.

13. The apparatus of claim 11, comprising one or more of a data memory, program memory, LED indicator, power monitoring circuit, and calibration mechanism.

14. An apparatus for monitoring diabetes, the apparatus comprising:
a computing device;
a physical activity level sensor to enable measurement of energy consumption of a patient;
a power source;
a real time clock to provide the time at which measurements were performed; and
a wireless transceiver to wirelessly transmit measurement data to an external communications device.

15. The apparatus of claim 14, wherein said communications device is to receive data from said monitoring device, and is to implement one or more of processing data, displaying data, analyzing data, manually entering data and transmitting the data to said server.

16. The apparatus of claim 14, comprising one or more of a data memory, program memory, LED indicator, power monitoring circuit, and calibration mechanism

17. An apparatus for monitoring diabetes, the apparatus comprising:
a computing device;
a blood glucose level sensor to enable measurement of blood glucose levels of a patient;
a physical activity level sensor to enable measurement of energy consumption of a patient;
a power source;
a real time clock to provide the time at which measurements were performed; and
a wireless transceiver to wirelessly transmit measurement data to an external communications device.

18. The apparatus of claim 17, wherein said communications device is to receive data from said monitoring device, and is to implement one or more of processing data, displaying data, analyzing data, manually entering data and transmitting the data to said server.

19. The apparatus of claim 17, wherein said blood glucose level sensor and said physical activity level sensor share at least one of elements selected from the group consisting of front-end circuitry, CPU, memory, power source and transceiver.

20. The apparatus of claim 17, comprising one or more of a data memory, program memory, LED indicator, power monitoring circuit, and calibration capability.

21. An apparatus for administering treatment, the apparatus comprising:
a computing device;
a medication administration mechanism for administrating a dosage of medication;
a medication dosage sensor to enable measurement of medication type and/or dosage administered to a patient;
a power source;
a real time clock to provide the time at which measurements were performed; and
a wireless transceiver to wirelessly transmit measurement data to an external communications device

22. The apparatus of claim 21, wherein said medication dosage sensor comprises a dosage administration mechanism selected from the group consisting of tip sensors, dosage setting knob, potentiometer, trigger, color code sensor, conductive code sensor and tactile sensor.

23. The apparatus of claim 21, wherein said communications device is to receive data from said monitoring device, and is to implement one or more of processing data, displaying data, analyzing data, manually entering data and transmitting the data to said server.

24. The apparatus of claim 21, comprising a data memory, program memory, LED indicator, power monitoring circuit, and calibration mechanism.

25. The apparatus of claim 21, wherein said medication administration mechanism is an insulin administration mechanism.

26. A method for monitoring diabetes, the method comprising:
measuring one or more patient parameters using a diabetes monitoring device, said monitoring device including at least a wireless transceiver, to enable data to be communicated between said monitoring device and a communication device, said communication device including at least a wireless transceiver;
transmitting measurement data to said communications device; and
processing said measurement data by said communications device.

27. The method of claim 26, comprising transmitting measurement data to a server.

28. The method of claim 27, comprising preparing a response to said received data, and sending said response to one or more selected destinations.

29. The method of claim 28 wherein said response includes one or more of providing advice, warnings, alerts, alarms, and information to a selected destination.

30. The method of claim 26 comprising interacting with said monitoring device.

31. The method of claim 26, wherein said measuring of one or more patient parameters includes monitoring one or more of blood glucose level, physical activity, energy intake and insulin dosage.
